(19) United States
(12) Patent Application Publication
(10) Pub. No.: US 2005/0238731 A1
(43) Pub. Date: Oct. 27, 2005

(54) Composition and method for treating the effects of diseases and maladies of the upper digestive tract

(76) Inventor: Stephen Holt, Mountain Lakes, NJ (US)

Correspondence Address: MUSERLIAN, LUCAS AND MERCANTI, LLP, 475 PARK AVENUE SOUTH, 15TH FLOOR, NEW YORK, NY, 10016, US

(21) Appl. No.: 11/024,092

(22) Filed: Dec. 28, 2004

Related U.S. Application Data
(60) Provisional application No. 60/532,854, filed on Dec. 29, 2003.

(51) Int. Cl. A61K 35/78; A61K 33/10; A61K 33/32

(52) U.S. Cl. 424/643; 424/729; 424/765; 424/686; 424/687; 424/757; 514/453; 424/756; 514/458; 514/763; 514/474

(57) ABSTRACT

A medicinal composition using nutrients herbs, botanicals and other natural substances for supporting structures and functions in the upper digestive tract, including but not limited to beneficial effects on the lining and functions of esophagus, stomach and duodenum and the treatment of disorders of the digestive tract that are related to excessive secretions of acid and pepsin which affect any tissues of the upper digestive tract. The basis of the invention and method of treatment focuses on the use of natural substances which exert several effects on body structure and function, including but not limited to: immediate neutralization of acid secreted by the human or animal stomach, antioxidant effects on tissues, complimentary actions for the eradication of Helicobacter pylori (H. pylori) or primary actions on the eradication of H. pylori, delayed effects on the reduction of the secretion of gastric acid by a mechanism of inhibition of the function of gastric proton pumps. A key factor in this patent is the use of natural substances to achieve a bimodal, chronological effect on acid reduction. The inventive formulations and the range of the ingredients provide an opportunity for initial neutralization of gastric acid followed by an inhibition of gastric acid secretion, as a function of this unique formulation of natural ingredients with bio pharmaceutical actions. This invention can be used as complimentary therapy with all acid reducing natural substances and pharmaceuticals. Its unique formulation will compliment any attempts to reduce the damaging effects of gastric acid and reduce the damaging effects of H. pylori infection of the upper digestive tract.

### CROSS-REFERENCE TO RELATED APPLICATIONS

This application claims the benefit of priority from U.S. Provisional Patent Application Ser. No. 60/532,854, filed Dec. 29, 2003, the disclosure of which is incorporated herein by reference.

### FIELD OF THE INVENTION

The present invention is directed to compositions and methods for treating the effects of diseases and maladies of the upper digestive tract due to but not limited to damage from acid, pepsin, H-pylori infection, and the taking of substances or drugs which may damage the lining of the upper digestive tract. The present invention also relates to formulations containing combinations of different natural ingredients which are useful as a primary treatment or as complementary treatment, i.e. increasing the beneficial effects of other treatments used for upper digestive diseases and disorders, and/or disease management strategies.

### BACKGROUND OF THE INVENTION

Beginning in prehistoric times, humans have attempted to treat every known type of illness and malady with naturally occurring products. Such products were initially in their natural state, such as leaves, berries, roots, tree cuttings and extracts. With the advance of science, and greater understanding of chemistry, humans have been able to synthetically produce and extract a great variety of bio pharmaceuticals which were previously unknown or unidentified.

The scientific community has taken an increased interest in discovering the various effects of remedies of natural origin. Extensive studies have been conducted into the efficacy of a great number of these products and the results have largely been positive. As a result, consumers around the world have begun to take interest in these products due to the scientific data supporting the validity of their efficacy.

Over the years, there have been numerous advances in the treatment of various digestive tract maladies. In spite of the therapeutic advances provided by H₂ blockers such as ranitidine and proton pump inhibitors (PPIs) such as omeprazole, and the like, further advances have been sought. In particular, there is a growing need in the art for treatments which are based at least in part on natural ingredients, nutraceuticals and the like. The present invention addresses this need.

### SUMMARY OF THE INVENTION

It is therefore an object of the present invention to provide formulations of dietary supplements which synergistically combine the advantages of selected dietary supplements.

The foregoing objects and advantages of the invention are illustrative of those that can be achieved by the present invention and are not intended to be exhaustive or limiting of the possible advantages which can be realized. Thus, these and other objects and advantages of the invention will be apparent from the description herein or can be learned from practicing the invention, both as embodied herein or as modified in view of any variation which may be apparent to those skilled in the art. Accordingly, the present invention resides in the novel methods, arrangements, combinations, compositions and improvements herein shown and described.

In accordance with these and other objects of the invention, a brief summary of the present invention is presented. Some simplifications and omissions may be made in the following summary, which is intended to highlight and introduce some aspects of the present invention, but not to limit its scope.

A first aspect of the invention includes a nutraceutical composition for supporting body structures and functions in the upper digestive tract. It includes an effective amount of
a) an antioxidant having gastric proton pump inhibiting effects;
b) an acid neutralizer; and
c) an antibacterial agent effective against Helicobacter pylori.
   Some of the key dietary supplements included in the compositions of the present invention include the antioxidant ellagic acid, the acid neutralizer fava bean flour and the antibacterial agent mastic gum, alone or in combination with zinc.

Another aspect of the invention includes methods of treating various digestive disorders such as heart burn, gastro-esophageal reflux disease, sour stomach, gastritis, duodenitis, esophigitis, conditions related to excessive secretion of acid, negative effects of acid secreted by the stomach, upper digestive tract of infection with the bacteria H. pylori. The methods include administering an effective amount of the novel compositions described herein to patient in need thereof.

A still further aspect of the invention includes methods of increasing the effectiveness of a gastrointestinal therapy by administering an effective amount of the compositions described herein with a gastrointestinal therapy.

Detailed descriptions of preferred exemplary embodiments adequate to allow those of ordinary skill in the art to make and use the invention follow in later sections.

According to a broad aspect of the invention, there is disclosed a composition for a safe and effective dietary supplement to support body structure and function in the upper digestive tract by selecting a dietary supplement that can be used for preventing and treating acid peptic disorders and H. pylori infection in the upper digestive tract. The invention can be used for treating said predetermined symptoms of an ailment and/or the ailment itself in the upper digestive tract by combining into a formulation of natural substances used for the treatment of an upper digestive ailment or the ailment itself.

It is a further object of this invention to propose that the dietary supplement can be used as complimentary therapy for the prevention and treatment and/or management of upper digestive disorders due to alterations in structure and functions of the upper digestive tract as a consequence of acid or peptic damage to the upper digestive tract and/or infection with the bacterium H. pylori and all consequences including but not limited to the potential consequences of structural damages to the esophagus or any structures of the upper digestive tract. In a further object of this invention, there is disclosed a method for improving the outcome of the use of several drugs that may be used for a similar purpose to the proposed invention which comprises the development of a dietary supplement in a complex formula and specific delivery systems. This aspect of the invention includes both primary treatment benefits of the dietary supplement combination, together with complimentary treatment benefit to commonly used pharmaceuticals that are available by prescription or over the counter sale for the management of upper digestive complaints that may involve damage from acid and/or pepsin and/or the damaging effects of non-steroidal anti-inflammatory drugs on the digestive tract (e.g. inflammation of the lining of the upper digestive tract or the occurrence of ulceration of varying degrees) and infection with H. pylori. Thus, there is disclosed a method for improving the efficacy of pharmaceuticals through the selection of a dietary supplement used for the treatment of predetermined symptoms of an ailment and/or the ailment itself and selecting a nutraceutical (dietary supplement) which is also used for treating said predetermined conditions or related symptoms of an ailment and the ailment itself. The invention also includes the combining of natural ingredients used in the invention with pharmaceuticals and other nutraceuticals which can then be combined and formulated into different oral delivery mechanisms including but not limited to dosage forms as solids, powders or liquid forms and administered to a person in need thereof. Such dosages are intended to cover capsules, caplets, liquids, additions to other vehicles of oral delivery systems and even dilution in homeopathic treatments. The specific formulations of the product or products which are proposed in this invention are meant to include all oral forms of delivery including but not limited to chewable tablets, liquids and the addition of any other substances that may enhance the overall formulation, in terms of excipients or additions or coatings or other acts during manufacture or formulation of dietary supplement products which can result from the proposed invention.

In the present invention, there are disclosed preparations for treating the symptoms of upper digestive upset as well as treating pain and discomfort associated with upper digestive symptoms (specifically including but not limited to heartburn), and all changes in body structure and function that can occur as a consequence of stomach acid secretion, pepsin secretion or H. pylori infection. The invention is specifically designed to either compliment or replace treatment with H₂ receptor antagonist drugs of all types in all doses and proton pump inhibitor drugs of all types and in all doses where viable indications exist for substitution and/or complimentary use when such drugs are used for their effects on upper digestive function and/or reduction in acid secretion and/or other mechanism of action, including but not limited to their ability to combat H. pylori infection or any of its consequences and or to combat the negative effects of non-steroidal anti inflammatory drugs of all types, including aspirin, on the digestive tract. The preferred ingredients in the invention are disclosed herein, but as will be appreciated by those of ordinary skill, pharmaceutical additions and/or the additions of other natural ingredients or substitutions of proposed formulations are also within the scope of the invention.

One of the advantages of the compositions of the present invention is the fact that these natural ingredients, when used in the combinations described herein, have particular bimodal or multi-modal effects on gastric acid, with immediate neutralizing effects, inhibitory effects on stomach acid secretion and other effects including but not limited to antioxidant effects, antagonistic effects on the damaging nature of acid, pepsin and all factors involved in H. pylori infection. It has been surprisingly found that significant therapeutic benefits are obtained when immediate acid neutralization effects are combined with preventative and healing effects in the treatment of digestive tract disorders. It is believed that this combination therapy, which is based largely on dietary supplements and natural products, provides superior results when compared to single mode therapies, i.e. acid neutralization alone, acid secretion alone, cytoprotective therapy alone or antibacterial therapy against H. pylori alone. The inventive compositions provide a useful alternative to polypharmaceutical approaches with greatly reduced concern for drug interactions and side effects which are common when multiple organically synthesized compounds are administered to patients.

### DETAILED DESCRIPTION OF THE PREFERRED EMBODIMENTS

For the purposes of this specification, the word “pharmaceutical” refers to a material that is:
a) a synthetically produced bioactive compound, where no structurally identical, naturally produced analog to the synthetically produced bioactive compound exists; or
b) a biologically active compound derived from a living organism, where the biologically active compound is not a dietary supplement.

The pharmaceuticals utilized in this invention include the equivalent and alternative salts which may be formulated from the base pharmaceuticals and which achieve substantially the same effect as the pharmaceutical listed.

For the purposes of this specification, a “dietary supplement” is defined as a product (other than tobacco) that bears or contains one or more of the following dietary ingredients: a vitamin, a mineral, an herb or other botanical, an amino acid, a dietary substance for use by man to supplement the diet by increasing the total daily intake of that substance, or a concentrate, metabolite, constituent, extract, or combinations of these ingredients.

The word “nutraceutical,” for the purposes of this specification, refers to a food item, or a part of a food item, that offers medical health benefits, including prevention and/or treatment of disease. More particularly, a nutraceutical is a material that is:
a) a dietary supplement containing a nutritive bioactive compound; or
b) a biologically active processed or unprocessed material derived from a plant, a fungus, an animal, or a portion thereof; where the precise composition of the biologically active processed or unprocessed material may be undetermined.

Examples of a biologically active processed material may include a finely chopped, powdered, pureed, or cooked material derived from plant or animal tissue, or an extract of plant or animal tissue.

The word “antibacterial” as used herein shall be understood to include primarily dietary supplements having demonstrated activity against H. pylori. It may also include pharmaceutical products such as metronidazle and/or tetracycline or other known agents effective against the bacteria in connection with or as a replacement the dietary supplement.

Pharmaceuticals for use in treating acid reflux disease or gastric ulcers or any acid peptic disease or disease resulting from H. pylori infection include the Histamine H₂-receptor antagonists Cimetidine, Famotidine, Nizatidine, and Ranitidine and others are particularly useful for this purpose, as are proton pump inhibitors such as omeprazole, esmeprazole and all other such safe and/or effective proton pump inhibitors. It is a clear part of this invention that components of the proposed dietary supplement products work in part by inhibiting gastric proton pumps and other components of the dietary supplements have acid neutralizing capabilities by virtue of their content of fava bean flour and other similar substances that can be derived from legumes or plants, used with or without standard types of mineral antiacids containing, but not limited to calcium and magnesium in all salt forms that can produce neutralization of acid. A key aspect of this invention is the use of substances that neutralize acidity in the upper digestive tract with immediacy as a result of the release of acid neutralizing substances in the formulation reacting with the acid in the upper digestive tract. This reaction preferably takes place with chewable forms of the inventive dietary supplement formulations and/or liquid forms and/or any forms that provide rapid dissolution such as wafers, delivery that can occur in the in the buccal cavity and subsequently swallowed to reach the upper digestive tract.

The therapeutic uses of the pharmaceuticals used in the present invention are well known and need no further explanation. The nutraceuticals which may be used in the present invention have a variety of medicinal uses which improve the efficacy of pharmaceuticals and/or can be substituted for pharmaceuticals in first line, complete or incomplete therapies of acid related disorders and H. pylori infection and other causes of upper gastrointestinal damage such as non steroidal anti-inflammatory drugs. The dietary supplement formulations which are proposed in the present invention can be used with or without any appropriately indicated pharmaceutical for any circumstances described within the disease or disorder parameters presented in this invention.

Antioxidants require special mention in this invention. This invention uses much information that shows that antioxidants can effectively prevent the progression of damage in the upper gastrointestinal tract that occurs as a result of acid, pepsin, H. pylori infection, the taking of any ulcerogenic drug or agent and any other mechanism of damage to the lining of the upper gastrointestinal tract that can be afforded directly or indirectly as a consequence of any ingredients in the proposed formulations. This invention includes all classes of antioxidants which are safe for human consumption and which can be administered as a dietary supplement or nutraceuticals. These antioxidants include the natural, sulfur-containing amino acid allicin, which acts to increase the level of antioxidant enzymes in the blood. Herbs or herbal extracts, such as garlic, which contain allicin are also effective antioxidants.

The catechins, and the extracts of herbs such as green tea containing catechins, are also effective antioxidants. Extracts of the immune boosters Astragalus membranaceous, Astragalus mongolicus, and other herbs of the genus Astragalus also show antioxidant activity. The bioflavonoids, such as quercetin, hesperidin, rutin, and mixtures thereof, are also effective as antioxidants. The primary beneficial role of the bioflavonoids may be in protecting vitamin C from oxidation in the body. This makes more vitamin C, or ascorbic acid, available for use by the body. Ascorbic acid, which is itself an important antioxidant nutraceutical, functions as a free radical scavenger that helps reduce oxidative stress and/or cell damage caused by free radicals.

Particularly important in this invention is the use of ellagic acid and salvianolic acid which are known in animal experiments to inhibit the function of gastric proton pumps that secrete acid. Furthermore, a body of opinion exists that antioxidants play a major role in the prevention of damage consequent upon H. pylori infection or the taking of ulcerogenic drugs including but not limited to non-steroidal anti-inflammatory drugs and aspirin. Furthermore, evidence exists that oxidative damage to the upper gastrointestinal tract may result in changes in cellular morphology, including but not limited to metaplasia (e.g. Barrett's Esophagus) which are states of pre-cancerous change which are documented to result in cancer of the upper gastrointestinal tract in several locations including but not limited to the esophagus and stomach. Therefore, the use of high dosages of antioxidants, such as ellagic acid, and other antioxidants are cancer chemo preventive properties of the invention. To date, there has been no disclosure of complimentary therapies used as adjuncts to existing pharmaceuticals or nutraceuticals for cancer chemo prevention within the remit and scope of this patent application. These matters are amplified in a further discussion in this patent submission and represent novel therapies concerning the individual and combined management of what has been termed “acid related disease” which includes but is not limited to dissolved disruptions of body structure and functions consequent upon acid damage, pepsin damage or any damage with or without H. pylori infection or the taking of ulcerogenic drugs in any form, be it parenteral or oral or by topical application.

Bioflavonoids such as quercetin are also effective anti-inflammatory agents, and may be used as such in the inventive compositions. Anti-inflammatory herbal nutraceuticals and anti-inflammatory nutraceutical compounds derived from plants or herbs may also be used as anti-inflammatory agents in the inventive compositions. These include bromolain, a proteolytic enzyme found in pineapple; teas and extracts of stinging nettle; turmeric, extracts of turmeric, or curcumin, a yellow pigment isolated from turmeric.

Liver protectants are also effective nutraceuticals which may be used in this invention. Silymarin, an extract from milk thistle seeds containing three isomeric flavonolignans, is a particularly effective liver protectant, and is useful in treatment of patients with AIDS. Milk thistle and its extracts also appear to exhibit some antioxidant activity.

Another nutraceutical which can be used in the present invention is ginger, derived from herbs of the genus Zingiber, such as Zingiber officinale, Zingiber capitatum and Zingiber zeruzmbet. This nutraceutical has been found to possess cardiotonic activity due to compounds such as gingerol and the related compound shogaol as well as providing benefits in the treatment of dizziness, and vestibular disorders. Ginger is also effective in the treatment of nausea and other stomach disorders.

Other nutraceuticals effective against stomach disorders are licorice and its extracts, and aloe vera. Licorice stimulates the bile production by the liver, and can relieve ulcers and stomach aches and lower cholesterol. Studies on animals indicate that aloe vera and extracts or juices prepared therefrom help maintain a healthy stomach lining and assist in digestion. L-glutamine is also effective in treating digestive disorders, as are juices containing L-glutamine. L-Glutamine helps protect the structural integrity of the bowels, making it useful for treating ulcers and “leaky gut syndrome.”

Although some of the sample nutraceuticals listed above have been described as to their pharmacological effects, other nutraceuticals may also be utilized in the present invention and their effects are well documented in the scientific literature.

The preferred ingredients of the dietary supplement formulations for preventing or treating or managing upper digestive disorders referred to in this patent including but not limited to acid related disease, H. pylori infection and ulcerogenic medications, drugs or substances such as alcohol, tobacco smoke or any other damaging agent that is ingested or absorbed in to the body that may cause upper digestive disturbance are listed in one formulation that has undergone open label observation for the control of the symptom heart burn in six volunteers with recurrent heart burn and this formulation was made in several batches of a chewable tablet with the following ingredients that have been modified from time to time. These chewable tablets contain the following ingredients with amounts listed in two chewable tablets. The range of the ingredients has been made in different dosage amounts of each ingredient and tested in at least two volunteers for each of six batches of varying dosage of ingredients. Each batch consistently relieved symptoms of mild moderate or severe heart burn in open labeled observations in volunteers with symptoms of gastresophageal reflux disease. Each batch of products when sampled did not result in any adverse effects with complete tolerance and acceptance of the taste and palatability of the various mixtures.

One preferred batch formulation includes the following ingredients and amounts in two chewable tablets:
Ellagic acid (Pomegranate & Raspberry Fruit), 350 mg,
raspberry Fruit 60 mg,
Green Tea 50% Polyphenols, 50 mg,
Turmeric Root, 10 mg,
Vitamin C, 60 mg, 100% RDI,
Vitamin E (d-Alpha Tocopherol Succinate), 30 IU, 100% RDI,
Vitamin A (Beta Carotene), 5000 IU, 100% RDI,
Zinc (Amino Acid Chelate, 15 mg, 100% RDI,
Calcium (carbonate), 200 mg, 20 5 RDI,
Magnesium (carbonate), 100 mg,
Sodium Bicarbonate up to about 300 mg,
3%, Fava Bean, 800 mg,
Mastic Gum, 40 mg,
Lecithin Powder, 40 mg,
Apple Pectin, 18 mg, and
gastric Mucin, 2 mg.
   The chewable tablets are prepared using standard tableting techniques known to those of ordinary skill.

In view of the above, some alternative formulations can include from 25% to 200% of the amounts shown above for the principle ingredients, antioxidants, acid neutralizers and anti-H. pylori agents with the ancillary ingredients being adjusted as needed, replaced or eliminated. It will be further understood that art customary colorants, flavorants, etc. can also be included in amounts well known to those of ordinary skill.

While the above formulations will provide a dosage containing an effective amount of the inventive combination of the dietary supplements, it is will be understood by those of ordinary skill that an “effective amount” shall be understood to refer to the amount needed to control achieve a desired therapeutic result. Depending upon its use, the compositions of the invention will administered on an as needed (prn) or suggested dosing schedule for a period of time. As those in the art will understand that the amounts required to achieve the desired result(s) will vary depending upon a number of factors. These factors include, for example, the person being treated, his weight, general physical condition, the type of digestive tract symptoms being treated, etc.

Without wishing to be bound by any particular theory, it is believed that effective amounts of the compositions of the invention can range from 1 to 4 or greater doses a day of compositions containing the following principal ingredients/dose:
Total Antioxidants: 10-2,000 mg, preferably 100-1,200 mg;
Total Acid Neutralizers: 5-3,000 mg, preferably 50-2,000 mg; and
Total Antibacterial against H. pylori: 1-1,000 mg, preferably 10-200 mg.

More particularly, preferred compositions can include per dose:
a) One or more of the following antioxidants in amounts within the range provided above: ellagic acid (preferred), salvianoic acid, allicin, catechins, Astragalus extracts, bioflavinoids ascorbic acid, vitamin E, vitamin A, and green tea polyphenols;
b) One or more of the following acid neutralizers in amounts within the range provided above: calcium carbonate, magnesium carbonate, magnesium hydroxide, aluminum hydroxides, sodium bicarbonate, and fava bean flour (preferred); and
c) One or more of the following anti-H. pylori agents in amounts within the range provided above: mastic gum or zinc.

In still further aspects of the invention, the nutraceutical compositions of the invention can include:

(I) FORMULA
a) from about 100 to about 800 mg Ellagic acid;
b) from about 0 to about 180 mg raspberry fruit;
c) from about 0 to about 200 mg Green Tea 50% polyphenols;
d) from about 0 to about 100 mg Turmeric Root;
e) from about 0 to about 2,000 mg Vitamin C;
f) from about 0 to about 90 IU Vitamin E (d-Alpha Tocopherol Succinate);
g) from about 0 to about 15000 IU Vitamin A (Beta Carotene);
h) from about 0 to about 60 mg Zinc (Amino Acid Chelate);
i) from about 0 to about 500 mg Calcium carbonate;
j) from about 0 to about 500 mg Magnesium carbonate;
k) from about 0 to about 180 mg Sodium Bicarbonate;
l) from about 200 to about 2400 mg Fava Bean flour;
m) from about 10 to about 120 mg Mastic Gum;
n) from about 0 to about 120 mg Lecithin Powder;
o) from about 0 to about 50 mg pectin; and
p) from about 0 to about 10 mg gastric Mucin.
   (II) FORMULA
a) from about 300 to about 400 mg Ellagic acid;
b) from about 20 to about 80 mg raspberry fruit;
c) from about 20 to about 80 mg Green Tea 50% polyphenols;
d) from about 4 to about 12 mg Turmeric Root;
e) from about 30 to about 150 mg Vitamin C;
f) from about 20 to about 40 IU Vitamin E (d-Alpha Tocopherol Succinate);
g) from about 3,000 to about 8,000 IU Vitamin A (Beta Carotene);
h) from about 10 to about 20 mg Zinc (Amino Acid Chelate);
i) from about 100 to about 300 mg Calcium carbonate;
j) from about 50 to about 150 mg Magnesium carbonate;
k) from about 100 to about 350 mg Sodium Bicarbonate;
l) from about 500 to about 1,000 mg Fava Bean flour;
m) from about 20 to about 60 mg Mastic Gum;
n) from about 0 to about 120 mg Lecithin Powder;
o) from about 0 to about 50 mg pectin; and
p) from about 0 to about 10 mg gastric Mucin.
   Reducing Acid Secretion and GERD

The management or treatment of GERD is directed at symptom relief, attempts to heal inflammation of the lining of the esophagus (esophagitis), and measures to prevent the complications of the disease. The mainstay of treatment of GERD has been to use drugs that can reduce the secretion of acid by the stomach. Acid in the stomach contents washes back into the esophagus and it is a major factor causing damage to the esophagus.

There are several drugs available by prescription or over-the-counter purchase that reduce acid secretion in a potent manner. The most effective of these drugs are called proton pump inhibitors because they block the “pumping” of acid by specialized cells in the stomach (parietal cells). Examples of commonly used proton pump inhibitors are omeprazole, lansoprazole and esomeprazole (Prilosec®, Prevacid® and Nexium®, respectively). Other popular drugs that reduce gastric acid secretion belong to a class of compounds called H₂-receptor antagonists. They include the drugs cimetidine, ranitidine, famotidine and nizatidine (Tagamet®, Zantac®, Pepcid® and Axid®, respectively).

While these acid-reducing drugs are safe and effective, they are expensive and the symptoms of GERD promptly return when people stop using them. These drugs are often equivalent in their ability to reduce symptoms overall, but the more potent, acid-reducing, proton pump inhibitor drugs (e.g. omeprazole) seem to be more effective at healing the inflammation in the esophagus caused by GERD (esophagitis).

Pumping Acid

The cells of the stomach that make gastric acid can be switched on and off by a variety of signals. Nerves and hormones provide these signals. At the top, or apex, of these acid-secreting cells is a complex series of pumps that work to push acid (hydrogen ions) out of these cells into the cavity of the stomach. These powerful pumps work against a gradient like a water pump emptying a swimming pool. These “pumps” are called the gastric proton pumps (gastric=stomach, proton=acid, H⁺ ions).

There are drugs and natural substances that inhibit the functions of these pumps (switch them off). These gastric proton pump inhibitors form a class of drugs that are the most popular treatments for peptic ulcers and gastroesophageal reflux diseases (GERD). The proton pump inhibitor drugs are well known to many people. They include omeprazole (Prilosec® and Nexium®). A number of natural inhibitors of gastric proton pumps have been identified, and among the most preferred herein are ellagic acid and salvianolic acid. Ellagic acid has its common origins in raspberries or pomegranates and is believed to block acid secretion in the stomach by interfering with the gastric proton pumps.

Properties of Ellagic Acid: an Antioxidant

Ellagic acids (elagitannins) are naturally occurring powerful antioxidants found in several fruits, especially raspberries and pomegranates. This substance is an example of the many “phenolic” compounds that occur in fruits and vegetables. Ellagic acid has many described health benefits. Scientific studies show that ellagic acid is an anti-cancer agent and an anti-inflammatory substance. It has blood pressure-lowering effects, a minor sedative action and it can inhibit the secretion of stomach acid by blocking the proton pumps (gastric H⁺, K⁺—ATPase exchange mechanisms).

Ellagic Acid and Oxidative Stress in the Gut

Before one examines the ability of ellagic acid to block the secretion of stomach acid, one must consider that there are several, highly desirable, putative actions of ellagic acid in the complimentary management of peptic ulcers and esophagitis (GERD). These added actions go beyond any ability of ellagic acid to alter acid secretion by the stomach. Oxidative stress due to free-radical damage is an important factor in the progression of esophagitis and peptic ulcer disease. Ellagic acid is a powerful antioxidant. Furthermore, H. pylori, the bacterium associated with peptic ulcer disease, creates much damage by causing oxidative stress to the lining of the gastrointestinal tract. Ellagic acid can help counter this oxidative stress caused to the lining of the upper digestive tract by H. pylori.

It is known that severe, long-standing esophagitis and gastric ulcer can lead to the development of cancer in the esophagus- and stomach, respectively. Again, this progression to the development of cancer from pre-existing inflammation (ulcer or esophagitis) may be related to oxidative stress with mutagenic (pre-cancerous) changes in the lining of the esophagus or stomach.

Ellagic acid may help counter these progressions to malignant change in the lining of the esophagus and stomach by its anti-cancer effects (inhibition of carcinogenicity and mutagenicity). Furthermore, the association of H. pylori infection with gastric cancer may be linked by chronic (long-term) oxidative damage to the stomach, and ellagic acid may inhibit these effects.

Ellagic Acid Inhibits Stomach Acid Secretion

Japanese and U.S. researchers have shown that ellagic acid could work to both suppress the secretion of stomach acid and inhibit the occurrence of ulcers in the stomach of animals that are caused by experimental “stress.” Moreover, these researchers examined the dosing schedules of ellagic acid that are required to achieve the effects of reduction in stomach acid secretion. Acid reducing actions of drugs or natural substances are potentially valuable in the treatment of GERD (esophagitis) and peptic ulcer in humans.

Effects of Ellagic Acid on the Gastric Proton Pump

The most popular medicines used in the treatment of GERD (esophagitis) and peptic ulcers are a class of drugs called “proton pump inhibitors.” Examples of these pharmaceuticals are omeprazole (Prilosec®), esomeprazole (Nexium®) and lansoprazole (Prevacid®). These drugs block the “gastric proton pumps” in an irreversible manner, and they produce significant reductions in acid secretion. This undoubtedly is valuable in the treatment of acid—peptic disease (esophagitis, GERD and peptic ulcers). However, it has been shown that ellagic acid can do the same thing in certain dosages; but in addition to reducing acid secretion, ellagic acid has desirable antioxidant, putative cancer-preventive and other beneficial actions that are not shared by the drug inhibitors of the gastric proton pumps (omeprazole, lansoprazole and esomeprazole).

A further beneficial effects of ellagic acid on the stomach is its demonstrated ability to reduce the occurrence of ulcers in the stomach as demonstrated using a rat model. These findings of a positive reduction in stomach ulcers and stomach acid secretions in the animals were compared with similar effects that can be caused by the popular ulcer/heartburn drug cimetidine (Tagamet®). It was apparent in this research that at a certain dosage level of ellagic acid stomach acid secretion was blocked; and ulcers could be prevented in part by the administration of ellagic acid.

Human Observation

Unfortunately, there have been no controlled clinical trials on the ability of ellagic acid to heal peptic ulcers or esophagitis in humans, but there are open label observations that ellagic acid has helped people with dyspepsia and heartburn, referred to earlier in this patent application. At the very least, ellagic acid is valuable as a complementary remedy in the management of upper digestive acid-reducing issues. Ellagic acid has beneficial effects in these disorders that go beyond its ability to reduce gastric acid secretion by blocking the proton pumps in the parietal cells of the stomach. These added beneficial effects included antioxidant and putative anti-cancer effects which have not been directly studied.

Development of Dietary Supplements with Acid Fighting H. pylori Fighting and Ulcerogenic Drug or Substance Fighting Properties

In an article discussing the effects of switching some anti-ulcer medications from prescription to over-the-counter availability (OTC), I highlighted the fact that early symptom relief drives the use of drugs that lower acid secretion (Holt S., “Over-the-counter histamine H₂-receptor antagonists. How will they affect the treatment of acid-related diseases?” Drugs 47 (1): 1-11, 1994). The popularity of simple antacids, such as Maalox® or Turns®, is due largely to the fact that their contents (alkalis) cause immediate neutralization of stomach acid and quick relief of symptoms. However, H₂ receptor antagonist drugs available OTC are used as effective heartburn remedies. This knowledge has resulted in the development of pharmaceuticals in which the rapid neutralizing capabilities of antacids are combined with the more delayed benefits of taking an acid-lowering medication (such as cimetidine, famotidine, ranitidine, etc.), such as Pepcid Complete®. In this non-prescription product, the combination of an antacid (rapid neutralizer) and acid-reducing drug (famotidine, Pepcid®) provides both fast and long-lasting relief from heartburn. This treatment modality can be enhanced when combined with the compositions of the present invention or avoided, if desired, by substituting the pharmacologic agent combination with the compositions of the present invention.

Good Scientific Agreement on Rapid and Lasting Approaches to Heartburn and Upper Digestive Symptoms

Ellagic acid can be used as a component of an acid fighting natural product which has the added advantage of a natural immediate neutralizer in the form of fava bean flour. Fava bean flour has many of the advantages of regular antacids in buffering acid without side effects.

Combining fava bean flour with ellagic acid is a natural approach that attempts to duplicate the approaches used in combinations of antiacids and acid-lowering drugs. The fava bean flour can rapidly neutralize gastric acid and help with heartburn, “sour stomach” and dyspepsia, and its component of ellagic acid inhibits the gastric proton pump potentially causing longer-lasting digestive relief from longer-lasting suppression of the secretion of stomach acid. The combination of fava bean flour with ellagic acid and other antioxidants provides an alternative to this pharmacologic approach to modulate gastric acidity and provide a synergistic activity in which acid secretion is reduced, acid content is neutralized and healing is initiated.

Fava Bean Flour and Salts of Calcium and Magnesium in a Preferred 2:1 Ratio or the Inclusion of Other Natural or Mineral Salt Antiacids

High acidity in the stomach (hyperacidity) has been managed for many years by the taking of antacids (acid neutralizers), such as sodium bicarbonate, magnesium hydroxide, calcium carbonate, aluminum hydroxide, etc. Each of these various inorganic antacids possesses disadvantages or limitations. Sodium bicarbonate is an excellent neutralizer of acid but its effects are short-lived and excessive sodium intake can occur. Excessive intake of sodium can raise blood pressure and exert other negative cardiovascular effects. Calcium carbonate can cause kidney problems when given in excess and excessive calcium intake can actually cause a rebound stimulation of gastric acidity (delayed hyperacidity). While magnesium causes a loose bowel movement, or even diarrhea, calcium tends to be constipating. Most people avoid aluminum-containing antacids because of the uncertainty of aluminum toxicity. The presence of aluminum deposits in the brain in degenerative nervous system diseases has frightened many people.

The search for an alternative to effective antacids in the form of Maalox®, Mylanta®, Tums®, Rolaids®, etc. is stimulated by the drawbacks with these inorganic antiacid “salts.” Fava beans can be ground to fine flour which has all the desirable characteristics of a natural antacid (a neutralizing substance for gastric acid). Studies in the laboratory show the clear immediate acid-neutralizing capacity of fava bean flour, and it has been reported to effectively manage simple heartburn and “sour stomach” when used as part of a dietary supplement.

Natural Inhibitors of Gastric Acid Secretion

There are many chemical substances in plants, fruits and vegetables that belong to a class of chemical compounds called phenols (polyphenols). Ellagic acid is only one example of these “phenolic” substances which are well characterized in other herbs or plants, such as green tea (polyphenols, such as epi-gallo-catechins). The phenolic compound salvianolic acid is known to be an inhibitor of the acid-producing proton pumps in the stomachs. Salvianolic acid can be isolated from the root of a plant used as a traditional Chinese medicine.

Salvianolic acid can be prepared from the roots of the plant Salvia miltiorrhize (Lamiaceae) and it has had uses in the treatment of coronary heart disease, as has ellagic acid. In the case of salvianolic acid, there seems to be a specific inhibition of the gastric proton pump in animal experiments and this natural substance also has anti-coagulant activity (interferes with blood clotting). Experiments using salvianolic acid in animals confirm its ability to reduce gastric acid secretion and reduce the formation of gastric ulcers in the stomach of animals that have had the exits to their stomachs blocked (pylorus-ligated animals). It also prevents the development of stomach ulcers in animals that have been exposed to the severe stresses of cold water immersion or restraint. The doses of salvianolic acid required to have an anti-ulcer effect are quite high and this may limit the general use of this compound in humans, in the absence of well constructed safety studies. In contrast, ellagic acid has much precedent of safe use in the food chain. It is found in many foods, including raspberries and pomegranates.

The power of ellagic acid in decreasing acid secretion by an action similar to proton pump inhibitor drugs is a potential revolution in the application of phytochemicals in dietary supplements. While natural reducers of stomach acid secretion may not be as potent as drugs, this may be an advantage. The value of retaining a healthy amount of acid in the stomach has been stressed in natural medicine. Standard antacids, such as chalk, magnesium salts and aluminum hydroxide, have their equivalents in “Nature,” e.g. fava bean flour. Much more research is required to define the role of natural neutralizers of stomach acid (fava bean flavor) and natural inhibitors of gastric acid secretion, such as ellagic acid.

Helicobacter Pylori

The bacterium Helicobacter pylori (HP) is clearly recognized as a major cause of chronic gastritis, peptic ulcer (stomach and duodenum) and the development of gastric cancer. Two Australian physicians Barry Marshal, M.D. and his colleague J. Robin Warren, M.D. discovered the association of H. pylori with “dyspepsia” in the early 1980s. Since then, scientists have toiled to explain the mechanisms of the damaging effects of H. pylori. H. pylori does not act like a virulent infectious agent. This bacterium results in an infection where the living bacteria sit beneath the mucus layer of the stomach. Helicobacter pylori goes about its business of creating havoc with the lining of the upper digestive tract, while it lives beneath the gastric mucus layer.

Helicobacter Pylori Causes Oxidative Stress

H. pylori causes immune responses which result in inflammatory processes to rid the body of the organism. A number of antibodies to H. pylori have been readily detected in the blood and measured by many scientists. The damaging effects of H. pylori relate to its ability to generate free radicals (or reactive oxygen species). Free radicals are discussed increasingly in conventional and alternative medicine as a primary cause of many acute and chronic diseases. The generation of free radicals causes oxidative stress to tissues and this can result in inflammation and sometimes even cancer.

Many popular healthcare and scientific books describe the importance of free radicals as generators of disease. An understanding of free radicals involves a look at the role of oxygen in body tissues. Oxygen is necessary to sustain life, but it can combine with body tissues to cause oxidation. This is the general example of the “free radical chain of oxidation,” a circumstance that causes unwanted effects on tissues called “oxidative damage” or “oxidative stress.” We have learned that natural phytochemicals, such as ellagic acid, anthocyanidins or polyphenols (found in colored fruits and vegetables), can fight free-radical damage and prevent oxidative stress.

A number of natural substances, such as vitamins C, A and E and a whole range of phytochemicals (phyto=plant), are powerful antioxidants. Examples of naturally occurring antioxidant substances found in plants include ellagic acid from raspberries or pomegranates, soy isoflavones, catechins in green tea and curcumin from turmeric.

The Link Between H. Pylori and Oxidative Stress: The Value of Antioxidants

It has been shown in laboratory experiments that adding H. pylori to stomach lining cells results in the generation of free radicals or reactive oxygen species. Predictably, levels of antioxidants such as vitamin C are found to be depleted in the stomach secretions of patients with gastritis. Vitamin C is mainly present in secretions from stomachs infected with H. pylori in a reduced or biologically inactive form.

Eradicating Helicobacter Pylori

The most important facet in the treatment of H. pylori infections is the eradication of the organism. The eradication of H. pylori has been associated with evidence of less free-radical generation. The eradication of H. pylori results in a rise of vitamin C levels in gastric juice. The use of antioxidants in H. pylori infection is straightforward, safe and potentially effective at reducing tissue oxidation. While some studies show that certain antioxidant vitamins (vitamins A and E) are found in normal amounts in H. pylori-infected tissues, the potential role of antioxidant therapy in acid peptic disease is emerging with great importance in alternative medicine.

Antioxidants, H. Pylori and Gastric Cancer: A Link?

This whole area of nutritional research on H. pylori infection requires greater scrutiny. It provides some very interesting options for the application of remedies of natural origin and helps explain why antioxidants may be preventive against cancer, or chemo-preventive. To illuminate this issue, we know that H. pylori is associated with a risk of gastric cancer. This risk may be due, in major part, to the damage to free radicals as induced by H. pylori infection. The anti-cancer benefits of green tea, soy and herbs like turmeric may be well explained by the actions of the antioxidants they contain, such as epi-gallo-catechin gallate (in green tea), ellagic acid (in raspberries), isoflavones, such as daidzein and genistein (in soy), and curcumin (in tumeric).

Long-standing H. pylori infection seems to be associated with changes in the lining cells of the stomach (metaplasia), resulting in pre-cancerous changes. Several phytochemicals, particularly green tea catechins and ellagic acid, have been shown to help prevent the occurrence of pre-malignant changes in cells (metaplasia). This is the anti-cancer, anti-mutagenic property of some powerful antioxidants. It is quite logical to propose that antioxidants could play a role in cancer prevention by their ability to counter oxidative stress. Therefore, antioxidants should be considered very valuable adjuncts (additives) to any medical attempts to manage bad changes in body tissues resulting from H. pylori infection. They are thus included as a key part of the nutraceuticals of the invention.

Drugs and Dyspepsia

The efficacy of H₂-receptor antagonists (Pepcid®, Zantac®, Tagamet®, etc.) in the treatment of “non-ulcer dyspepsia” is variable and sometimes disappointing. Individuals who have predominantly “reflux” type, or GERD-like, symptoms are a group who may respond favorably to nutraceuticals containing fava bean flour and ellagic acid.

Non-Steroidal Anti-Inflammatory Drugs (NSAID): Damage to the Upper Digestive Tract

Anyone with miserable joint pain or the pain of an acute injury has enjoyed the temporary benefit provided by non-steroidal anti-inflammatory drugs, or NSAID (e.g. Aspirin, Ibuprofen®, Naproxen®, Suldinac®, Advil®, Alleve®, etc). Furthermore, many pain sufferers have experienced the short-term relief afforded by acetaminophen (Tylenol®). Practitioners of alternative medicine have criticized some conventional physicians concerning the use of NSAID or aspirin. Alternative medical concepts stress the availability of effective natural alternatives that can replace or complement the use of NSAID or aspirin (www.arthotrim.com). Innovations in dietary supplement development have revealed the effectiveness of chondro protection (protecting cartilage in joints) with glucosamine; and there are natural inhibitors of Cox-2 enzymes that cause inflammation in joints affected arthritis (www.arthrotrim.com, www.supraflex.com). NSAID have severe side effects in a significant number of people. The side effects of NSAID include bleeding from the upper or lower digestive tract, liver toxicity and reductions of renal (kidney) function. It is frequently stated that NSAID are only a “quick-fix” approach because they neither alter the clinical course of arthritis, nor go to the root of the disorder. Some NSAID may actually damage joints in some cases. This further damage induced by some NSAID has been shown to occur in animals. Furthermore, NSAIDs may contribute to “leaky guts” and aspects of the present invention may contribute to the avoidance of damage to both the upper mid and lower digestive tracts that may occur from the use of ulcerogenic drugs such as NSAID's. It is therefore another aspect of the invention that compositions include the nutrceuticals described herein in combination with an NSAID whereby the damaging effects of the NSAID are minimized by the concomitant present of the dietary supplements described herein.

Specific Aspects Concerning Some Key Ingredients in the Formulation

The formulation given in only one example is not meant to limit the range or constituents that can be used within the remit of this patent application. In brief review, ellagic acid and related compounds, regardless of source are used primarily for their proton pump inhibiting effects that will reduce, modulate or modify gastric acid secretion, but this activity may be shared by other anti oxidants, especially those with related physico-chemical characteristics and/or pharmacodynamic actions. Furthermore, antioxidants are cancer chemo preventive e.g. ellagic acid and green tea polyphenols, constituents mentioned with some degree of specificity in this patent application. In order for antioxidants to be effective in tissues antioxidants of different redox potential have to be used with different degrees of hydro- or lipophilic characteristics. This is an important focus of the formulation which reinforces the claim of lack of restriction of selection of certain antioxidants which in the example proposed include, but are not necessarily meant to be limited to, turmeric or curcuminoids, vitamins C, E, A or their variants and other antioxidant vitamins or minerals with direct or indirect antioxidant function in the body (e.g. selenium, not provided in the example). The inclusion of zinc together with antioxidants is particularly relevant to assistance in the eradication or management or amelioration of the damaging effects of H. pylori or ulceroginc drugs or acid or pepsin secretion or any other factors of disturbed physiology that result in changes in body structure and function in the upper digestive system. The inclusion of salts of calcium, magnesium and sodium are primarily for their antiacid effects, but hey have other beneficial effect on digestive function.

Mastic gum has been described as killing H. pylori even in low dosages. This resinous exudates obtained from the stem and the main leaves of Pistacia Lentiscus has been used as a food ingredient in the Mediterranean region for thousands of years. Clinically, mastic has been effective in the treatment of benign gastric ulcers and duodenal ulcers. In rats, mastic showed cytoprotective and mild antisecretory properties. Mastic killed the H. pylori NCTC 11637 strain and the six clinical isolates (reduction in the viable count by a factor of 1000) irrespective of the organism's level of susceptibility to nitroimidazoles . . . these results suggest that mastic has definite antibacterial activity against H. pylori (Huwez F U, Thirwell D, New England Journal of Medicine, December 1998 and Huwez F U, Al Habbal M J, Gatroenterol Japon 1986; 21:273-4). It must be emphasized that the formulations proposed are purposely complex and differing in dosages of ingredients in order to result in a synergistic effect, meaning that components have additive benefits in their bio pharmacological actions.

Lecithin powder can help provide building blocks for mucosal integrity by nutritional means and pectins and/or gastric mucin can neutralize acid or pepsin or provide mucosal protective effects in the upper digestive tract.

Fava bean flour is a food based supplement that has been used for the relief of indigestion, heart burn or sour stomach or other digestive symptoms. Fava bean flour can be shown to neutralize stomach acid and it meets test requirements of an antiacid in USP standards or in tests established by the Food and Drug administration of the US for over the counter anitacids. The fava bean is a major food source in southern Europe and the Middle East, extending to Northern Africa.

The statements made herein are supported with good scientific agreement in peer reviewed scientific literature and the demonstration of synergy among these components of formulations and modifications thereof which have been shown to be effective in the relief of heart burn and sour stomach in open label observations. As such, this is compelling evidence of a synergistic effect for symptom relief or upper digestive problems and further science exists to support the other claims of this and other combinations of natural ingredients which will deal with the immediate, intermediate or delayed consequences of acid damage, pepsin damage, ulcerogenic drug damage or H. pylori induced damage to the upper gastrointestinal tract.

The inventive compositions and methods described herein are designed specifically to make several inclusive claims concerning the beneficial management, treatment, cure or prevention of upper digestive complaints and disorders of body structure and function that result from the direct or indirect damage caused by adverse lifestyle, such as poor nutrition and substance abuse and other factors such as acid pepsin or H. pylori infection or the taking of ulcerogenic drugs or substances, affecting the upper digestive tract including but not limited to the esophagus, stomach and duodenum. The invention is particularly applicable to the condition of reflux of acid from the stomach into the esophagus, commonly referred to as gastro-esophageal reflux disease and its variance of functional upper gastrointestinal disorders, sometimes referred to as GERD, reflux, dyspepsia, upper abdominal pain or discomfort, acid regurgitation, heart burn and other medical terms or terms in common use to describe organic or functional disorders of the upper digestive tract within the scope proposed within this invention, a unique aspect of this claim is the combination of early acid inhibition with more delayed acid inhibition of stomach acid in locations of the upper digestive tract. In addition, the proposed formulations have specific actions on oxidative stress and secondary inflammatory responses that cause disruption of the lining of the upper gastro intestinal tract and all of its consequences including but not limited to inflammation of the lining of the gastrointestinal tract, the development of ulceration or breaches in the lining of the digestive tract and any consequential damage or occurrence such as narrowing the digestive tract (e.g. stricture) or the development of cancer or other tissue changes which are pre malignant or pre cancerous.

While the example of one formulation has been specifically given the dosages and amounts of the ingredients within the proposed formulations can vary by as much as 200% difference in dosage because of their general precedency for safety and inclusion in dietary supplements which have been defined by the DSHEA of 1994 in the US. This invention is not limited to the proposals in the dietary and health education act of 1994. While specific examples are given, they are not given to limit the scope of the formulation or the applications of the invention to the disorders described in this patent application. Although the present invention has been described in detail with particular reference to preferred embodiments thereof, it should be understood that the invention is capable of other different embodiments, and its details are capable of modifications in various obvious respects. As is readily apparent to those skilled in the art, variations and modifications can be affected while remaining within the spirit and scope of the invention. Accordingly, the foregoing disclosure, description, and tables are for illustrative purposes only, and do not in any way limit the invention, which is defined only by the claims.

1. A nutraceutical composition for supporting body structures and functions in the upper digestive tract, comprising an effective amount of
a) an antioxidant having gastric proton pump inhibiting effects;
b) an acid neutralizer; and
c) an antibacterial agent effective against Helicobacter pylori.

2. The nutraceutical composition of claim 1, wherein the antioxidant is selected from the group consisting of ellagic acid, salvianoic acid, allicin, catechins, Astragalus extracts, bioflavinoids ascorbic acid, vitamin E, vitamin A, green tea polyphenols and mixtures thereof.

3. The nutraceutical composition of claim 2, wherein the antioxidant is ellagic acid.

4. The nutraceutical composition of claim 1, wherein the acid neutralizer is selected from the group consisting of calcium carbonate, magnesium carbonate, magnesium hydroxide, aluminum hydroxides, sodium bicarbonate, fava bean flour and combinations thereof.

5. The nutracetitical composition of claim 1, wherein the antibacterial agent effective against Helicobacter pylori is selected from the group consisting of zinc, mastic gum and mixtures thereof.

6. A nutraceutical composition of claim 1 containing:
a) from about 100 to about 800 mg Ellagic acid;
b) from about 0 to about 180 mg raspberry fruit;
c) from about 0 to about 200 mg Green Tea 50% polyphenols;
d) from about 0 to about 100 mg Turmeric Root;
e) from about 0 to about 2,000 mg Vitamin C;
f) from about 0 to about 90 IU Vitamin E (d-Alpha Tocopherol Succinate);
g) from about 0 to about 15000 IU Vitamin A (Beta Carotene);
h) from about 0 to about 60 mg Zinc (Amino Acid Chelate);
i) from about 0 to about 500 mg Calcium carbonate;
j) from about 0 to about 500 mg Magnesium carbonate;
k) from about 0 to about 700 mg Sodium Bicarbonate;
l) from about 200 to about 2400 mg Fava Bean flour;
m) from about 10 to about 120 mg Mastic Gum;
n) from about 0 to about 120 mg Lecithin Powder;
o) from about 0 to about 50 mg pectin; and
p) from about 0 to about 10 mg gastric Mucin.

7. A nutraceutical composition of claim 6 containing:
a) from about 300 to about 400 mg Ellagic acid;
b) from about 20 to about 80 mg raspberry fruit;
c) from about 20 to about 80 mg Green Tea 50% polyphenols;
d) from about 4 to about 12 mg Turmeric Root;
e) from about 30 to about 150 mg Vitamin C;
f) from about 20 to about 40 IU Vitamin E (d-Alpha Tocopherol Succinate);
g) from about 3,000 to about 8,000 IU Vitamin A (Beta Carotene);
h) from about 10 to about 20 mg Zinc (Amino Acid Chelate);
i) from about 100 to about 300 mg Calcium carbonate;
j) from about 50 to about 150 mg Magnesium carbonate;
k) from about 100 to about 350 mg Sodium Bicarbonate;
l) from about 500 to about 1,000 mg Fava Bean flour;
m) from about 20 to about 60 mg Mastic Gum;
n) from about 0 to about 120 mg Lecithin Powder;
o) from about 0 to about 50 mg pectin; and
p) from about 0 to about 10 mg gastric Mucin.

8. The nutraceutical of claim 1, further comprising a pharmacologic agent selected from the group consisting of proton pump inhibitors, H₂ blockers and combinations thereof.

9. A method of supporting the body structures and functions in the upper digestive tract in a mammal, comprising administering an effective amount of the composition of claim 1 to a patient in need of such therapy.

10. A method of treating a digestive disorder selected from the group consisting of heart burn, gastro-esophageal reflux disease, sour stomach, gastritis, duodenitis, esophigitis, conditions related to excessive secretion of acid, negative effects of acid secreted by the stomach, upper digestive tract of infection with the bacteria H. pylori, comprising administering an effective amount of a composition of claim 1 to patient in need thereof.

11. A method of increasing the effectiveness of a gastrointestinal therapy, comprising administering an effective amount of the composition of claim 1 in combination with a gastrointestinal therapeutic agent.

12. The method of claim 11, wherein the gastrointestinal therapeutic agent is a proton pump inhibitor.
